(19) United States
(12) Patent Application Publication
(10) Pub. No.: US 2026/0234512 A1
(43) Pub. Date: Aug. 13, 2026

(54) MICROCAPSULES

(71) Applicant: IBERCHEM S.A.U., Alcantarilla Murcia (ES)

(72) Inventors: Michael John Robert WHITE, Alcantarilla Murcia (ES); Sandra CERMEÑO OLMOS, Alcantarilla Murcia (ES); Sandra GALLEGO TORNEL, Alcantarilla Murcia (ES); Ginés MARTÍNEZ SÁNCHEZ, Alcantarilla Murcia (ES)

(73) Assignee: IBERCHEM S.A.U., Alcantarilla Murcia (ES)

(21) Appl. No.: 19/117,277

(22) PCT Filed: Oct. 13, 2023
(86) PCT No.: PCT/EP2023/078515

§ 371 Date: Mar. 31, 2025

(30) Foreign Application Priority Data
Oct. 25, 2022 (GB) 2215786.1

(51) Int. Cl. C11D 3/50; B01J 13/16; B01J 13/22; C11D 3/16; C11D 3/37

(52) U.S. Cl. CPC C11D 3/505; B01J 13/16; B01J 13/22; C11D 3/162; C11D 3/3726

(57) ABSTRACT

The present invention relates to microcapsules comprising a hydrophobic core within a polymeric shell, wherein a) the polymeric shell is formed from shell components comprising: i) a polyisocyanate, wherein the polyisocyanate is selected from xylylene diisocyanate (XDI) and oligomers, adducts and derivatives thereof; ii) a polycaprolactone polyol comprising from 2 to 4 free hydroxyl groups; iii) a polyethyleneimine; iv) optionally, other shell components; and b) the hydrophobic core comprises an active material. The invention further relates to a slurry comprising the microcapsules, a home care formulation comprising the microcapsules, a personal care formulation comprising the microcapsules and a process for producing the microcapsules.

### FIELD OF THE INVENTION

The present invention relates to microcapsules comprising an active core material within a polymeric shell, a home care formulation or personal care formulation comprising the microcapsules and a process of forming the microcapsules.

### BACKGROUND

Microencapsulation systems are known for encapsulating active core materials such as fragrance materials. The encapsulation process results in a microcapsule comprising a core of active material surrounded by a polymer shell. Often the active material is hydrophobic which allows the shell to be polymerized around particles (for example, droplets) of the hydrophobic core which are dispersed and/or emulsified in aqueous medium and/or solvent.

Various methods for making core-and-shell microcapsules have been proposed in the literature. For instance, it is known to encapsulate a hydrophobic core substance by dispersing the core substance into an aqueous medium containing a melamine formaldehyde (MF) pre-condensate and then reducing the pH to produce a microcapsule comprising an aminoplast resin shell wall surrounding the core substance. EP2794839B discloses aminoplast microcapsules which are stabilized by a polyisocyanate. Such aminoplast microcapsules are not generally considered to be biodegradable.

A need exists to provide improved microcapsules or to address one or more disadvantages of the prior art.

### SUMMARY OF THE INVENTION

The present invention is based in part on the surprising recognition by the inventors that the combination of shell components in the microcapsules according to the invention may help to improve the biodegradability and/or performance of the capsules when compared with other microcapsules.

Viewed from a first aspect, the present invention provides microcapsules comprising a hydrophobic core within a polymeric shell, wherein:
a) the polymeric shell is formed from shell components comprising:
   i) a polyisocyanate, wherein the polyisocyanate is selected from xylylene diisocyanate (XDI) and oligomers, adducts and derivatives thereof;
   ii) a polycaprolactone polyol comprising from 2 to 4 free hydroxyl groups;
   iii) a polyethyleneimine; and
   iv) optionally, other shell components; and
b) the hydrophobic core comprises an active material.

Viewed from a second aspect, the invention provides a slurry comprising microcapsules of the first aspect, water and at least one surfactant.

Viewed from a third aspect, the invention provides a home care formulation or a personal care formulation comprising a slurry of the second aspect or microcapsules of the first aspect.

Viewed from a fourth aspect, the invention provides a process of producing microcapsules according to the first aspect, wherein the process comprises the steps of:
a) forming a polymerization system comprising an aqueous phase and a dispersed oil phase, wherein the oil phase comprises said active material, said polyisocyanate shell component and said polycaprolactone polyol shell component;
b) reacting the shell components by adding said polyethyleneimine shell component to the aqueous phase to form microcapsules comprising a core of the oil phase within the polymeric shell;
c) optionally, adding a deposition additive to the surface of the microcapsules; and
d) optionally, neutralizing the microcapsules using a metal hydroxide or inorganic acid.

Viewed from a fifth aspect, the invention provides microcapsules obtainable by a process according to the fourth aspect.

Any aspect of the invention may include any of the features described herein regarding that aspect of the invention or any other aspects of the invention.

### DETAILED DESCRIPTION OF THE INVENTION

In the context of the present invention, microcapsules are understood to be microparticles comprising at least one or more active material(s) as core material inside the capsule and which are enclosed by a polymeric capsule shell or capsule wall. The active materials are preferably hydrophobic or lipophilic ingredients. Such ingredients are preferably insoluble or poorly soluble in water, but readily soluble in fats and oils.

It will be understood that any upper or lower quantity or range limit used herein may be independently combined.

It will be understood that, when describing the number of carbon atoms in a substituent group (e.g. ‘C1 to C6’), the number refers to the total number of carbon atoms present in the substituent group, including any present in any branched groups. Additionally, when describing the number of carbon atoms in, for example fatty acids, this refers to the total number of carbon atoms including the one at the carboxylic acid, and any present in any branch groups.

Many of the chemicals which may be used to produce the present invention are obtained from natural sources. Such chemicals typically include a mixture of chemical species due to their natural origin. Due to the presence of such mixtures, various parameters defined herein can be an average value and may be non-integral.

The term biodegradability is the ability of organic material to be degraded to water, carbon dioxide (CO₂), and biomass after a prescribed time under defined conditions of temperature, oxygen, and moisture in the presence of microorganisms or fungi. According to the standard test method under OECD 301F, a test material (e.g. microcapsule shell) is considered readily biodegradable if more than 60% of the test material (e.g. microcapsule shell) has degraded after 28 days.

The term ‘personal care formulation’ when used herein means a consumer product intended to be applied to the human body or any part thereof for cleansing, beautifying, or improving appearance. Personal care formulations include but are not limited to cosmetics; deodorants; bar soaps; liquid soaps; facial and body washes; facial and body cleansers; hair shampoos; hair conditioners; toothpastes; shaving creams or gels; and foot care products. A personal care formulation does not include any product for which a prescription is required.

The term ‘home care formulation’ when used herein means a consumer product for use by household and/or institutional consumers for cleaning, caring, or conditioning of the home. Home care formulations include but are not limited to detergents including laundry detergents and dishwashing detergents; conditioners including fabric conditioners; cleaning formulations including hard surface cleaners; polishes and floor finishes.

The term “polymerization system” when used herein means the aqueous phase, oil phase, shell components and all other ingredients used in producing the microcapsules.

### Microcapsules

Microcapsules according to the invention comprise a hydrophobic core within a polymeric shell, wherein:
a) the polymeric shell is formed from shell components comprising:
   i) a polyisocyanate, wherein the polyisocyanate is selected from xylylene diisocyanate (XDI) and oligomers, adducts and derivatives thereof;
   ii) a polycaprolactone polyol comprising from 2 to 4 free hydroxyl groups;
   iii) a polyethyleneimine; and
   iv) optionally, other shell components; and
b) the hydrophobic core comprises an active material.

The microcapsules may be produced in a polymerization system. A slurry may comprise the produced microcapsules, water and at least one surfactant. Preferably the microcapsules do not comprise an aminoplast resin. As used herein, an aminoplast resin is a urea-formaldehyde (UF) or a melamine-formaldehyde (MF) resin. Both types of aminoplast resin may be undesirable for environmental reasons, such as poor biodegradability.

Particle size parameters of the microcapsules (e.g. D10, D50 or D90 volume mean diameter) may be measured by laser diffraction particle size analysis. The measurement may be made using a Malvern Mastersizer 3000 E with the measurement cell Hydro EV.

The microcapsules may have a D10 volume mean diameter (i.e. the point below which 10% of the microcapsules are contained, measured on a volume basis as described herein) of at least 0.5 μm, preferably at least 1 μm, more preferably at least 1.5 μm. The microcapsules may have a D10 volume mean diameter of at most 30 μm, preferably at most 20 μm, more preferably at most 10 μm.

The microcapsules may have a D50 volume mean diameter, measured as described herein, of at least 2 μm, preferably at least 4 μm, more preferably at least 5 μm. The microcapsules may have a D50 volume mean diameter of at most 50 μm, preferably at most 40 μm, more preferably at most 30 μm, yet more preferably at most 20 μm.

The microcapsules may have a D90 volume mean diameter, measured as described herein, of at least 7 μm, preferably at least 9 μm, particularly at least 11 μm. The microcapsules may have a D90 volume mean diameter of at most 80 μm, preferably at most 60 μm, particularly at most 40 μm.

Shell Component i)—Polyisocyanate

A first component of the polymeric shell is a polyisocyanate. The polyisocyanate is selected from xylylene diisocyanate (XDI) and oligomers, adducts and derivatives thereof. Surprisingly, a benefit of using XDI or an oligomer, adduct or derivative thereof in microcapsule production is that such polyisocyanates have one or more of the following advantages: excellent degree of reactivity, an improved pot life, improved heat resistance and/or reduced tendency of the polymer to yellowing. The polyisocyanate may be selected from xylylene diisocyanate (XDI), hydrogenated XDI (H6XDI), XDI-TMP (trimethylolpropane) adduct, XDI trimer and H6XDI-TMP adduct. Such polyisocyanates are available from Mitsui Chemicals under the Takenate series of products. Preferably the polyisocyanate is selected from an XDI trimer (such as Takenate D131N ex Mitsui) or an XDI-TMP adduct (such as Takenate D110N ex Mitsui). Preferably the polyisocyanate is selected from an XDI trimer, an XDI-TMP adduct and mixtures thereof. The polyisocyanate may be an XDI trimer. The polymeric shell may comprise only one polyisocyanate.

Preferably the polymeric shell does not comprise isophoronediisocyanate (IPDI).

The polymeric shell may comprise 20% to 75% by weight of the polyisocyanate, on the basis of the total weight of shell components in the microcapsules. The polymeric shell may comprise at least 25%, preferably at least 30% by weight of the polyisocyanate, on the basis of the total weight of shell components in the microcapsules. The polymeric shell may comprise at most 70%, preferably at most 65%, more preferably at most 60% by weight of the polyisocyanate, on the basis of the total weight of shell components in the microcapsules.

The amount of polyisocyanate in the microcapsules may also be specified by reference to the amount of polyisocyanate included in the polymerization system, as in Example 1 below. The polymerization system may comprise at least 0.5 wt % of polyisocyanate on the basis of the total weight of the polymerization system, preferably at least 1 wt %, more preferably at least 1.5 wt %, particularly at least 2 wt %. The polymerization system may comprise at most 8 wt % of polyisocyanate on the basis of the total weight of the polymerization system, preferably at most 6 wt %, more preferably at most 4 wt %.

Shell Component ii)—Polycaprolactone Polyol

A second component of the polymeric shell is a polycaprolactone polyol comprising from 2 to 4 free hydroxyl groups. The polycaprolactone polyol may be a diol, triol or tetrol, preferably a diol or triol, more preferably a triol. Preferably the polycaprolactone polyol is formed from caprolactone monomer and a diol (for example diethylene glycol or butanediol) or triol (for example trimethylolpropane) initiator. Without being bound by theory, the presence of the polycaprolactone polyol in the polymeric shell may surprisingly increase the biodegradability of the polymeric shell or microcapsules, preferably when measured using the method described in OECD 301F.

The polycaprolactone polyol may have a molecular weight (preferably number average molecular weight) of at least 200 g/mol, preferably at least 300 g/mol, more preferably at least 350 g/mol. The polycaprolactone polyol may have a molecular weight (preferably number average molecular weight) of at most 20,000 g/mol, preferably at most 15,000 g/mol, more preferably at most 10,000 g/mol, yet more preferably at most 5,000 g/mol, preferably at most 4,000 g/mol, preferably at most 3,000 g/mol, preferably at most 2,000 g/mol, preferably at most 1,500 g/mol. Preferably the polycaprolactone polyol has a molecular weight (preferably number average) in the range from 200 to 10,000 g/mol, more preferably from 300 to 5,000 g/mol, more preferably from 300 to 2,000 g/mol.

Suitable polycaprolactone polyols include, but are not limited to, the CAPA range available from Perstorp/Ingevity, and mixtures thereof. The polycaprolactone polyol may be selected from CAPAs 2043, 2101, 2201, 2205, 2209, 2201A, 2203A, 2302, 2402, 7201A, 7203, 3031, 3050, 3091 and 4101 ex Perstorp/Ingevity, preferably the polycaprolactone polyol is selected from CAPA 2043, CAPA 2101 and CAPA 3050 ex Perstorp/Ingevity. Preferably the polycaprolactone polyol is a polycaprolactone triol.

The polymeric shell may comprise 0.5% to 50% by weight of the polycaprolactone polyol, on the basis of the total weight of shell components in the microcapsules. The polymeric shell may comprise at least 1%, preferably at least 2.5% by weight of the polycaprolactone polyol, on the basis of the total weight of shell components in the microcapsules. The polymeric shell may comprise at most 40%, preferably at most 30%, more preferably at most 20% by weight of the polycaprolactone polyol, on the basis of the total weight of shell components in the microcapsules. Preferably the polymeric shell comprises 2.5% to 30% of the polycaprolactone polyol, by weight, on the basis of the total weight of shell components in the microcapsules,

The amount of polycaprolactone polyol in the microcapsules may also be specified by reference to the amount of polycaprolactone polyol included in the polymerization system, see Example 1 below. The polymerization system may comprise at least 0.05 wt % of polycaprolactone polyol on the basis of the total weight of the polymerization system, preferably at least 0.1 wt %, more preferably at least 0.15 wt %, particularly at least 0.2 wt %. The polymerization system may comprise at most 2 wt % of polycaprolactone polyol on the basis of the total weight of the polymerization system, preferably at most 1 wt %, more preferably at most 0.75 wt %, particularly at most 0.5 wt %.

Shell Component iii)—Polyethyleneimine

Another component of the polymeric shell is a polyethyleneimine. Any molecular weight and any degree of crosslinking or branching of this polymer can be used in the present invention. Preferably the polyethyleneimine has a branched structure. Preferably the polyethyleneimine is not a linear polyethyleneimine. The polyethyleneimine may have a molecular weight (preferably number average molecular weight) of at least 500 g/mol, preferably at least 1,000 g/mol, more preferably at least 1,500 g/mol. The polyethyleneimine may have a molecular weight (preferably number average molecular weight) of at most 10,000 g/mol, preferably at most 8,000 g/mol, more preferably at most 6,000 g/mol, yet more preferably at most 5,000 g/mol, preferably at most 4,000 g/mol, preferably at most 3,000 g/mol, preferably at most 2,500 g/mol. Preferably the polyethyleneimine has a molecular weight (preferably number average) in the range from 500 to 5,000 g/mol. Preferably the polyethyleneimine is cationic. A cationic polyethyleneimine may advantageously facilitate retention of the microcapsules on fibrous surfaces e.g. fabrics or hair. Preferably the polyethyleneimine is cationic and has a branched structure.

Suitable polyethyleneimines are available from BASF (Ludwigshafen, Germany) under the LUPASOL grades (e.g., Lupasol FG, Lupasol G20 waterfree, Lupasol PR 8515, Lupasol WF, Lupasol FC, Lupasol G20, Lupasol G35, Lupasol G100, Lupasol G500, Lupasol HF, Lupasol PS, Lupasol HEO 1, Lupasol PN50, Lupasol PN60, Lupasol PO100 and Lupasol SK). Preferably the polyethyleneimine is LUPASOL PR 8515.

Preferably the polymeric shell comprises at least 0.5% by weight of the polyethyleneimine, on the basis of the total weight of shell components in the microcapsules, more preferably at least 1%, yet more preferably at least 1.5%, particularly at least 2%. Preferably the polymeric shell comprises at most 20% by weight of the polyethyleneimine, on the basis of the total weight of shell components in the microcapsules, more preferably at most 15%, yet more preferably at most 10%. Preferably the polymeric shell comprises 1% to 10% of the polyethyleneimine, by weight, on the basis of the total weight of shell components in the microcapsules.

The amount of polyethyleneimine in the microcapsules may also be specified by reference to the amount of polyethyleneimine included in the polymerization system, for example see Example 1 below. The polymerization system may comprise at least 0.05 wt % of polyethyleneimine on the basis of the total weight of the polymerization system, preferably at least 0.1 wt %, more preferably at least 0.15 wt %. The polymerization system may comprise at most 3 wt % of polyethyleneimine on the basis of the total weight of the polymerization system, preferably at most 2 wt %, more preferably at most 1 wt %, particularly at most 0.5 wt %.

### Optional Other Shell Components

In addition to the polyisocyanate, polycaprolactone polyol and polyethyleneimine, the polymeric shell may optionally comprise one or more other shell components. Preferably the optional shell components comprise an alkyl silicate. Preferably the optional shell components comprise a sugar or sugar alcohol. Preferably the polymeric shell further comprises at least one of an alkyl silicate, a sugar and a sugar alcohol. The polymeric shell may further comprise an alkyl silicate. The polymeric shell may further comprise a sugar or a sugar alcohol, preferably a monosaccharide, preferably glucose. Preferably the polymeric shell further comprises iv) an alkyl silicate; and v) a sugar or a sugar alcohol.

Preferably the polymeric shell further comprises an alkyl silicate. Preferably the alkyl silicate is polymeric. Preferably the alkyl silicate is ethyl silicate, more preferably an ethyl silicate polymer. The alkyl silicate may be selected from Wacker TES 40 WN and Dynasylan 40. The alkyl silicate may react during the formation of the polymeric shell to provide a polymeric silica (SiO₂) structure in the shell. The fast speed of this reaction may provide an advantage during production of the microcapsules. The microcapsules may comprise a polymeric silica structure. The polymeric shell may comprise a polymeric silica structure. The combination of polyisocyanate and alkyl silicate in the shell components may advantageously provide beneficial characteristics to the microcapsules. The alkyl silicate may increase the heat resistance of the microcapsules. The alkyl silicate may provide stability to the microcapsule slurry formed during production of the microcapsules. Without being bound by theory the alkyl silicate may provide stability to the microcapsules when they are present in a formulation which comprises the microcapsules and one or more surfactants. The surfactant(s) may be selected from anionic, cationic, non-ionic and zwitterionic surfactants, preferably anionic and cationic surfactants. The alkyl silicate may surprisingly improve the tolerance of the microcapsules to such surfactants. Many home care and personal care formulations include such surfactants, for example fabric detergents and fabric softeners.

Preferably the polymeric shell comprises at least 1% by weight of the alkyl silicate, on the basis of the total weight of shell components in the microcapsules, more preferably at least 2.5%, yet more preferably at least 5%, particularly at least 7.5%. Preferably the polymeric shell comprises at most 35% by weight of the alkyl silicate, on the basis of the total weight of shell components in the microcapsules, more preferably at most 25%. Preferably the polymeric shell comprises 7.5% to 25% of the alkyl silicate, by weight, on the basis of the total weight of shell components in the microcapsules.

The amount of alkyl silicate in the microcapsules may also be specified by reference to the amount of alkyl silicate included in the polymerization system, for example see Example 1 below. The polymerization system may comprise at least 0.2 wt % of alkyl silicate on the basis of the total weight of the polymerization system, preferably at least 0.4 wt %, more preferably at least 0.6 wt %. The polymerization system may comprise at most 4 wt % of alkyl silicate on the basis of the total weight of the polymerization system, preferably at most 3 wt %, more preferably at most 2 wt %.

Preferably the polymeric shell further comprises a sugar or a sugar alcohol (including but not limited to glucose and sorbitol), preferably a monosaccharide, more preferably glucose, particularly preferably glucose monohydrate. The sugar or sugar alcohol may react with the polyisocyanate to be incorporated as a shell component in the polymeric shell.

Preferably the polymeric shell comprises at least 5% by weight of the sugar or sugar alcohol, on the basis of the total weight of shell components in the microcapsules, more preferably at least 10%, yet more preferably at least 15%, particularly at least 20%. Preferably the polymeric shell comprises at most 60% by weight of the sugar or sugar alcohol, on the basis of the total weight of shell components in the microcapsules, more preferably at most 50%, even more preferably at most 40%.

The amount of sugar or sugar alcohol in the microcapsules may also be specified by reference to the amount included in the polymerization system, for example see Example 1 below. The polymerization system may comprise at least 0.1 wt % of sugar or sugar alcohol on the basis of the total weight of the polymerization system, preferably at least 0.5 wt %, more preferably at least 1 wt %. The polymerization system may comprise at most 5 wt % of sugar or sugar alcohol on the basis of the total weight of the polymerization system, preferably at most 4 wt %, more preferably at most 3 wt %.

Preferably the optional other shell components comprise at least one polymer. Such polymers include, for example polyamines and polyquaterniums. In certain embodiments, the at least one polymer may be selected from amphoteric and cationic polymers having a weight average molecular weight in the range of from 1,000 to 1,000,000 g/mol, preferably from 10,000 to 500,000 g/mol.

Preferably the polymeric shell does not comprise polylysine.

### Microcapsule Core

The microcapsule core comprises an active material. The core is hydrophobic overall. Preferably the active material is hydrophobic.

The active material may be a volatile material or a non-volatile material. Non-volatile actives provide technical benefits which differ from those coming from volatile actives such as fragrances. Non-volatile actives do not rely on olfactory perception but instead provide other effects. In this specification a “non-volatile material” means an active material that does not volatilise too much. A perfume or fragrance is not non-volatile. When applied to a surface and left at 25° C. a non-volatile material will lose less than 50% of its mass over a time of 7 days. A non-volatile active material typically has a boiling point greater than 250° C.

Preferably, the active material is selected from the group consisting of fragrances, perfumes, flavors, UV absorbers, emollient oils, insecticides, phase change materials (PCMs), dyes, inks, conditioning agents (e.g. hair or skin conditioning agents), cleaning agents (e.g. hair or skin cleaning agents), cosmetic actives, personal care actives, home care actives, pharmaceutical actives, agrochemical actives, oxidizing agents, bleaching agents, medicines, fertilizers, nutrients, enzymes, liquid crystals, catalysts, and chemical reactants. The active material may not comprise a pesticide.

More preferably, the active material is selected from fragrances, perfumes, flavors, UV absorbers, emollient oils, insecticides, phase change materials (PCMs), dyes, inks, conditioning agents (e.g. hair or skin conditioning agents), cleaning agents (e.g. hair or skin cleaning agents), cosmetic actives, personal care actives, home care actives, pharmaceutical actives and agrochemical actives. Preferably the active material is selected from fragrances, perfumes, flavors. Preferably the active material comprises a fragrance.

Preferably the active material is a volatile material. Preferably the volatile material will, when applied to a surface and left at 25° C., lose more than 50% of its mass over a time of 7 days.

The core may comprise a mixture of active materials. Preferably the active material is not a surfactant. The core may further comprise one or more oil-soluble diluents or solvents.

The core may comprise at least 40% of active material by weight on the basis of the total weight of the core, preferably at least 60%, particularly at least 70%, desirably at least 80% and especially at least 90%. The core may comprise 100% of active material by weight on the basis of the total weight of the core. The core may consist essentially of the active material.

Preferably, the active material is a mixture of at least one fragrance compound and at least one solvent. The nature and type of the fragrance compounds present in the microcapsules do not warrant a detailed description here, which in any case would not be exhaustive, the skilled person being able to select them on the basis of its general knowledge and according to the intended use or application and the desired organoleptic effect. Typically, the fragrance material is a mixture of fragrance compounds. The fragrance compounds may belong to chemical classes as varied as alcohols, aldehydes, ketones, esters, ethers, acetates, nitriles, terpenoids, nitrogenous or sulphurous heterocyclic compounds and essential oils, and said materials can be of natural or synthetic origin. Many of these fragrance compounds are listed in reference texts such as the book by S. Arctander, Perfume and Flavor Chemicals, 1969, Montclair, New Jersey, USA, or its more recent versions, the relevant parts of which are incorporated herein by reference.

Preferably the fragrance material comprises at least one fragrance compound selected from:
i) hydrocarbons;
ii) aliphatic alcohols;
iii) aliphatic ketones and oximes thereof;
iv) aliphatic carboxylic acids and esters thereof;
v) acyclic terpene alcohols;
vi) acyclic terpene aldehydes and ketones;
vii) cyclic terpene alcohols;
viii) cyclic terpene aldehydes and ketones;
ix) cyclic alcohols;
x) cycloaliphatic alcohols;
xi) cyclic and cycloaliphatic ethers;
xii) (ethoxymethoxy)cyclododecane;
xiii) cyclic ketones;
xv) esters of cyclic alcohols;
xvi) esters of cycloaliphatic carboxylic acids;
xvii) aromatic and aliphatic alcohols;
xviii) esters of aliphatic alcohols and aliphatic carboxylic acids;
xix) aromatic and aliphatic aldehydes;
xx) aromatic and aliphatic ketones;
xxi) aromatic and aliphatic carboxylic acids and esters thereof;
xxii) nitrogen-containing aromatic compounds;
xxiii) phenols, phenyl ethers and phenyl esters;
xxiv) heterocyclic compounds;
xxv) lactones; and
xxvi) essential oils.

Preferably the microcapsule core comprises at least one solvent. The solvent may assist the encapsulation of the active material by assisting the active material to remain in the core phase during polymerization of the shell. The solvent may comprise at least one ester, preferably an ester oil.

The solvent may be a hydrophobic material that is miscible with the fragrance compounds. The solvent may provide at least one of the following benefits: i) increase the compatibility of compounds in the active material, ii) increase the overall hydrophobicity of the core, iii) influence the vapor pressure of the core, and iv) provide rheological structure to the core. Suitable solvents are those having reasonable affinity for the fragrance compounds. The affinity may be determined by using a group contribution method to predict a partition co-efficient which can be expressed by a ClogP value. Preferably the solvent has a ClogP greater than 2.5, preferably greater than 3.5 and more preferably greater than 5.5. It should be noted that selecting a solvent and an overall active material with high affinity for each other will result in an improvement in the stability of the core.

It is preferred that the compounds in the active material have a ClogP of 0.5 to 15. Preferably the active material has a weight-averaged ClogP of at least 2. The use of fragrance compounds to make a fragrance material with a weight-averaged ClogP of at least 2 is likely to be suitable for encapsulation. The active compounds are generally water-insoluble, and may be delivered through the microcapsules of this invention onto consumer products in different stages such as damp and dry fabric. Without encapsulation, free fragrance compounds may evaporate or dissolved in water during use, e.g., during a wash cycle. Higher ClogP fragrance compounds are generally well delivered from a regular (non-encapsulated) fragrance in a consumer product, but are also suitable for encapsulation for overall fragrance character purposes, longer lasting fragrance delivery, or overcoming incompatibility with the consumer product. For example, active compounds that would otherwise be instable, cause thickening or discoloration of the product or otherwise negatively affect desired consumer product properties can be encapsulated to overcome such disadvantages.

The amount of active material in the microcapsules may be specified by reference to the amount of active material included in the polymerization system, for example see Example 1 below. The polymerization system may comprise at least 10 wt % of active materials on the basis of the total weight of the polymerization system, preferably at least 15 wt %, more preferably at least 20 wt %, particularly at least 25 wt %. The polymerization system may comprise at most 45 wt % of active materials on the basis of the total weight of the polymerization system, preferably at most 40 wt %, more preferably at most 35 wt %, particularly at most 30 wt %.

### Deposition Additive

Preferably the microcapsules further comprising a deposition additive on their surface. The deposition additive may be polymeric. Preferably the deposition additive is cationic. A cationic deposition additive may assist the deposition of the microcapsules on fibrous surfaces such as textiles or hair. Preferably the deposition additive comprises a quaternary nitrogen group. Preferably the deposition additive is a polyquaternium, more preferably polyquaternium-11 (available as Luviquat PQ11 from BASF). The deposition additive may be selected from Luviquat PQ11, Lupamin 9030, Salcare SC60, SoftCAT SX 1300 X, Jaguar C17, Merquat 550. The deposition additive may be added to the polymerization system during the process of making the microcapsules, preferably after the microcapsules have been formed. Preferably the polymerization system is heated after the deposition additive is added to bind the deposition additive to the surface of the microcapsules. The deposition additive may comprise a hydrolysed protein.

The polymerization system may comprise at least 2 wt % deposition additive on the basis of the total weight of the polymerization system, preferably at least 4 wt %, more preferably at least 6 wt %, particularly at least 8 wt %. The polymerization system may comprise at most 20 wt % deposition additive on the basis of the total weight of the polymerization system, preferably at most 18 wt %, more preferably at most 16 wt %, particularly at most 14 wt %.

### Polymerization System

Preferably the microcapsules are produced in a polymerization system. The polymerization system may comprise an aqueous phase and an oil phase. The oil phase may be a dispersed and/or emulsified oil phase. The aqueous phase, oil phase, shell components and all other ingredients used in the process of forming the microcapsules will be referred to herein as the ‘polymerization system’.

One or more components of the microcapsule may be present in the oil phase. Preferably the active material is present in the oil phase. Preferably the polyisocyanate is present in the oil phase. Preferably the polycaprolactone polyol is present in the oil phase. Preferably the alkyl silicate (when included) is present in the oil phase.

During formation of the microcapsules, the shell components are reacted to form the polymeric shell around the core. Reacting the shell components preferably forms microcapsules comprising a core of the oil phase within a polymeric shell. The core comprises the fragrance material.

The polymerization system may comprise a sugar or sugar alcohol (including but not limited to glucose and sorbitol), preferably a monosaccharide, more preferably glucose, particularly preferably glucose monohydrate. Preferably the sugar or sugar alcohol is present in the aqueous phase. The sugar or sugar alcohol may react with the polyisocyanate to be incorporated as a shell component in the polymeric shell of the microcapsules.

The polymerization system may further comprise one or more emulsifiers and/or other surfactants. An emulsifier, which may have a high HLB (preferably HLB of 10 to 20, more preferably 15 to 20), may be present in the aqueous phase to assist emulsification of the oil phase.

The polymerization system may further comprise at least one additive to assist the production of the microcapsules. The additive may comprise a hydrophilic polymer, for example a polymer containing pendant hydroxyl groups, for instance a polyvinyl alcohol. The polyvinyl alcohol may be present in the aqueous phase. The polyvinyl alcohol may be used in aqueous solution. The polyvinyl alcohol may be derived from polyvinyl acetate, and preferably between 75 and 99% of the vinyl acetate groups are hydrolyzed to vinyl alcohol units.

The polymerization system may comprise a carboxyalkylcellulose, preferably carboxymethylcellulose, particularly sodium carboxymethylcellulose.

The polymerization system may comprise at least 0.1 wt % sugar or sugar alcohol on the basis of the total weight of the polymerization system, preferably at least 0.5 wt %, more preferably at least 1 wt %. The polymerization system may comprise at most 8 wt % sugar or sugar alcohol on the basis of the total weight of the polymerization system, preferably at most 6 wt %, more preferably at most 4 wt %.

The polymerization system may comprise at least 0.05 wt % carboxymethylcellulose on the basis of the total weight of the polymerization system, preferably at least 0.1 wt %.

The polymerization system may comprise at most 2 wt % carboxymethylcellulose on the basis of the total weight of the polymerization system, preferably at most 1.5 wt %, more preferably at most 1 wt %.

The polymerization system may comprise at least 1 wt % urea on the basis of the total weight of the polymerization system, preferably at least 2 wt %. The polymerization system may comprise at most 8 wt % urea on the basis of the total weight of the polymerization system, preferably at most 6 wt %, more preferably at most 4 wt %.

The polymerization system may comprise at least 0.1 wt % xanthan gum on the basis of the total weight of the polymerization system, preferably at least 0.15 wt %. The polymerization system may comprise at most 2 wt % xanthan gum on the basis of the total weight of the polymerization system, preferably at most 1.5 wt %, more preferably at most 1 wt %.

Preferably the microcapsules of the invention are produced in the form of a slurry. The slurry may comprise the microcapsules, water and at least one surfactant.

An advantage of the microcapsules of the invention is that they are biodegradable, preferably readily biodegradable. The biodegradability of the microcapsules, preferably of the polymeric shell, may be measured by the standard manometric respirometry method described in OECD 301F. The polymeric shell, when tested for biodegradability using the method described in OECD 301F, preferably has a mean degradation value of at least 60% after 28 days, preferably at least 70%, more preferably at least 80%, yet more preferably at least 90%. The polymeric shell may have a mean degradation value of at most 100%, preferably at most 99% after 28 days.

Preferably the microcapsules do not comprise a wax with a melting point above 40° C.

Preferably the microcapsules do not comprise an aminoplast resin.

### Process of Forming the Microcapsules

A process of forming microcapsules according to the invention comprises the steps of:
a) forming a polymerization system comprising an aqueous phase and a dispersed oil phase, wherein the oil phase comprises said active material, said polyisocyanate shell component and said polycaprolactone polyol shell component;
b) reacting the shell components by adding said polyethyleneimine shell component to the aqueous phase to form microcapsules comprising a core of the oil phase within the polymeric shell;
c) optionally, adding a deposition additive to the surface of the microcapsules; and
d) optionally, adjusting the pH of the microcapsules using a metal hydroxide or inorganic acid.

The polymerization system may comprise a polyvinyl alcohol. Preferably the polymerization system does not comprise polyvinyl alcohol. The polymerization system may comprise a sugar or a sugar alcohol (including but not limited to glucose and sorbitol), preferably a monosaccharide, more preferably glucose, particularly preferably glucose monohydrate. The polymerization system comprise a carboxymethylcellulose, preferably sodium carboxymethylcellulose. The polymerization system may comprise any of the features of a polymerization system described herein. Once the microcapsules have been formed by polymerization, one or more optional post-polymerization steps may be taken.

Preferably the process comprises step c) adding a deposition additive to the surface of the microcapsules. Preferably the deposition additive is a cationic polymer. Preferably the deposition additive comprises a hydrolysed protein. The deposition additive may comprise any of the features of a deposition additive described herein.

If acidic shell monomers are used, the resulting microcapsules or microcapsule slurry may be acidic. Such microcapsules may be neutralized by the use of a hydroxide, preferably a metal hydroxide, more preferably an alkali metal hydroxide, particularly sodium hydroxide. Examples of suitable alkali metal hydroxides are NaOH and KOH. The microcapsules may also be neutralized by using an amine, for example ammonia, monoethanolamine, diethanolamine or triethanolamine, preferably ammonia. Alternatively, if the pH is too high, the microcapsules may be neutralized using an inorganic acid, preferably hydrochloric acid.

Preferably the process comprises step d) neutralizing the microcapsules using a metal hydroxide or inorganic acid. Preferably the pH of the microcapsules is adjusted to between 7 and 8. Preferably the metal hydroxide is NaOH and/or the inorganic acid is hydrochloric acid.

The process may optionally comprise the step of adding urea after the microcapsules have been formed. The urea may improve the curing efficiency and/or the physico-chemical and isolation properties of the microcapsules.

The process may optionally comprise the step of adding xanthan gum after the microcapsules have been formed. The xanthan gum may increase viscosity of the slurry and improve stability of the microcapsules, for example by preventing separation of the slurry.

### Formulations Comprising the Microcapsules

The microcapsules of the invention may be included in formulations with many different applications such as crop care formulations, health care formulations, pharmaceutical formulations, personal care formulations and home care formulations.

According to one aspect, the invention provides a personal care formulation comprising microcapsules or a slurry according to the invention. Preferably the personal care formulation is for topical application to skin or hair.

The personal care formulation may be selected from hand soaps; bar soaps; liquid soaps; facial and body washes; personal care cleansers; shampoos; conditioners; toothpaste; shaving creams or gels; foot care products, moisturizers, sunscreens, after sun products, body butters, gel creams, high perfume containing products, perfume creams, baby care products, hair treatments, hair colourants, skin toning and skin whitening products, water-free products, anti-perspirant and deodorant products, tanning products, 2-in-1 foaming emulsions, multiple emulsions, preservative free products, mild formulations, scrub formulations e.g. containing solid beads, silicone in water formulations, pigment containing products, sprayable emulsions, cosmetics, colour cosmetics, conditioners, shower products, foaming emulsions, make-up remover, eye make-up remover, and wipes. Preferably the personal care formulation is selected from hair care products, skin care products, cosmetics, personal care cleansers, deodorants and anti-perspirants.

The personal care formulation preferably comprises microcapsules or a slurry according to the invention and at least one additional personal care ingredient. The personal care ingredient may be selected from a cleaning agent, hair conditioning agent, hair styling agent, anti-dandruff agent, hair growth promoter, perfume, sunscreen, sunblock, pigment, moisturizer, film former, hair color, make-up agent, thickening agent, emulsifier, humectant, emollient, antiseptic agent, deodorant active, dermatologically acceptable carrier, surfactant, abrasive, absorbent, fragrance, colorant, essential oil, astringent, anti-acne agent, anti-caking agent, anti-foaming agent, anti-oxidant, binder, enzyme, enzyme inhibitor, enzyme activator, coenzyme, botanical extract, ceramide, buffering agent, bulking agent, chelating agent, cosmetic biocide, external analgesic, substantivity increasing agent, opacifying agent, pH adjuster, reducing agent, sequestrant, skin bleaching and/or lightening agent, skin conditioning agent, skin soothing and/or healing agent, skin treating agent, vitamin or preservative. Preferably the personal care ingredient is selected from a cleaning agent, hair conditioning agent, skin conditioning agent, hair styling agent, antidandruff agent, hair growth promoter, perfume, sunscreen compound, pigment, moisturizer, film former, humectant, alpha-hydroxy acid, hair colour, make-up agent, thickening agent, antiseptic agent, deodorant, surfactant. The personal care formulation may comprise microcapsules according to the invention and at least one surfactant. The at least one surfactant may be selected from anionic, cationic, non-ionic and zwitterionic surfactants, preferably anionic and cationic surfactants.

According to one aspect, the invention provides a home care formulation comprising microcapsules or a slurry according to the invention. Preferably the home care formulation is for application to fabric or textile.

The home care formulation may be selected from fabric detergents (in liquid, powder, concentrated, unit dose or tablet form), fabric softeners (in liquid, powder, concentrated, unit dose or tablet form), fabric wash additives, fabric scent boosters (in liquid, gel, tablet, powder or granule form), refresher sprays, air care products, cleaning products, fabric cleaners, fabric conditioners, stain removers, hard surface cleaners, hand dishwashing detergents, machine dishwashing detergents, polishes and floor finishes. Preferably the home care formulation is selected from fabric conditioners, fabric detergents, fabric softeners, fabric wash additives, fabric scent boosters, refresher sprays, air care products and cleaning products. Preferably the home care formulation is a fabric detergent or a fabric softener.

The home care formulation preferably comprises microcapsules or a slurry according to the invention and at least one additional home care ingredient. The home care ingredient may be selected from surfactants, builders, chelating agents, dye transfer inhibiting agents, dispersants, enzymes, enzyme stabilizers, catalytic materials, bleach activators, hydrogen peroxide, sources of hydrogen peroxide, preformed peracids, polymeric dispersing agents, soil removal/anti-redeposition agents, brighteners, suds suppressors, dyes, fabric softeners, carriers, structurants, hydrotropes, processing aids, solvents and/or pigments and mixtures thereof, preferably the home care ingredient is selected from the group consisting of surfactants, builders, chelating agents, fabric softeners. The home care formulation may comprise microcapsules according to the invention and at least one surfactant. The at least one surfactant may be selected from anionic, cationic, non-ionic and zwitterionic surfactants, preferably anionic and cationic surfactants.

Any or all the features described herein, and/or any or all of the steps of any method or process described herein, may be used in any combination in any aspect of the invention.

### EXAMPLES

The invention is illustrated by the following non-limiting examples. It will be understood that all test procedures and physical parameters described herein have been determined at atmospheric pressure and room temperature (i.e. about 25° C.), unless otherwise stated herein, or unless otherwise stated in the referenced test methods and procedures. All parts and percentages are given by weight unless otherwise stated.

### Test Methods

In this specification, the following test methods have been used:
(i) Particle size analysis (including measurement of D10, D50, D90 volume mean diameters) of the microcapsules was performed using a Malvern Mastersizer 3000E with supplied software and the measurement cell Hydro EV. This is a laser diffraction particle size analysis equipment which uses the Mie theory and the refractive index of the sample to determine the particle size distribution. The microcapsule sample is thoroughly mixed and then diluted into water for the particle size measurements to be taken. Various particle size parameters and distributions are automatically measured.
(ii) Fragrance release performance of the microcapsules in a fabric softener and in a liquid laundry detergent was evaluated using a towel washing protocol and scoring by panellists. The fabric softener and liquid detergent were made according to standard formulations to which a controlled amount of microcapsules were added. One towel per sample is hand washed (at 40° C.) in the formulation containing the microcapsules. All towels are evaluated after drying for 24 hours. The aim of these evaluations is to determine the effectiveness of the fragrance release by the microcapsules after rubbing the dry towel. The panellists recorded their score for fragrance performance before rubbing and after rubbing. The scoring system was from 1 to 10 with a higher score indicating a higher fragrance intensity and better performance. The detailed procedures for the evaluation of the fabric softener and liquid detergent are given below:

### Fabric Softener:

1) Depending on the number of panellists, 10 g of sample are prepared and used for each towel.
2) Dilute 10 grams of fabric softener into 1 litre at 40° C.
3) Stir with a spatula to dissolve product evenly through the water.
4) Put the towels in and rub the towels around 5 times against each other (washer should wear disposable gloves) and let it soak for 25 minutes.
5) Once the time has been completed, the towels are wrung out and left to dry for 24 hours before being evaluated (Hang out towel in an unperfumed atmosphere to dry).

### Liquid Laundry Detergent:

The method is like the softener one with the difference that after step 4, and when the 25 minutes are over, the towels are rinsed (5 times) with 1 litre of water at 40° C. for each towel used.
(iii) Biodegradability/biodegradation was determined by method OECD 301F as described in the document “OECD Guidelines for the Testing of Chemicals: Ready Biodegradability” (adopted by the Council of the OECD on Jul. 17, 1992). Method OECD 301F is a standard manometric respirometry test. According to OECD 301F, a test material is considered readily biodegradable if more than 60% of the test material has degraded after 28 days.

### Comparative Example A

Comparative microcapsules not according to the invention (Comparative Microcapsules A) were synthesised using the materials listed in Table 1.

TABLE 1
Polymerization system for Comparative Microcapsules A

| | | Amount |
|---|---|---|
| | | (wt % of total |
| | | polymerization |
| Material | Description | system) |
| Fragrance 1 | Fragrance compounds & solvents | 29.70 |
| Takenate D131N | Polyisocyanate (XDI trimer) | 2.15 |
| Wacker TES40 | Ethyl silicate polymer | 1.00 |
| Water | | 53.18 |
| Poval 18-88 | Polyvinyl alcohol | 0.34 |
| Finnfix 5 | Sodium Carboxymethylcellulose | 0.15 |
| Lupasol PR8515 | Polyethyleneimine | 0.20 |
| NaHCO₃ | Sodium bicarbonate | 0.33 |
| Luviquat PQ11 | Polyquaternium D11 | 10.00 |
| | (Deposition additive) | |
| Urea | | 2.75 |
| Keltrol RD | Xanthan gum | 0.20 |
| | TOTAL | 100.00 |

Comparative Microcapsules A were formed using the materials of Table 1 as follows. Pre-mix I was prepared from 4.42 g of Poval 18-88, 600.60 g of water and 1.95 g of Finnfix 5 with heating, if necessary, until completely dissolved. Pre-mix II was prepared from 386.10 g of Fragrance 1 (containing standard fragrance compounds and solvents available from Iberchem), 27.95 g of Takenate D131N (ex Mitsui Chemicals) and 13.00 g of Wacker TES40 (ex Wacker), added dropwise. The two pre-mixes I & II were combined and emulsified with the help of an Ultraturrax T25 at room temperature at a speed of 9000 rpm. The pH of the emulsion was then adjusted to 2.5 using aqueous hydrochloric acid solution (10% strength by weight). Then, at 35° C. and with stirring at 150 l/min, a solution of 2.60 g of Lupasol PR8515 (polyethyleneimine, ex BASF) in a sodium bicarbonate solution at 7% in water was added to the emulsion over the course of 2 hours. The reaction mixture was then subjected to the following temperature program: heating to 55° C. after those 2 hours, maintaining this temperature for 2 hours, then 3 hours at 80° C. After this time, add the 130.0 g of Luviquat PQ11 (ex BASF) and allow to mix for 15 minutes with Ultraturrax T25 at 8000 rpm. Then prepare a premix of urea and water (35.75 g of each) with heat at 60° C., add it to the mixture and when it dissolves add 2.6 g of Keltrol RD to the mixture with stirring and turn off the heating plate. The mixture was then cooled to room temperature. Finally, the pH of the produced microcapsule slurry was adjusted to 7.5 using aqueous sodium hydroxide solution or formic acid solution.

The produced microcapsule slurry will be referred to as Comparative Microcapsules A.

### Example 1

Microcapsules according to the invention (Microcapsules 1) were synthesised using the materials listed in Table 2.

TABLE 2
Polymerization system for Microcapsules 1

| | | Amount |
|---|---|---|
| | | (wt % of total |
| | | polymerization |
| Material | Description | system) |
| Fragrance 1 | Fragrance compounds & solvents | 29.70 |
| Takenate D131N | Polyisocyanate (XDI trimer) | 2.15 |
| Wacker TES40 | Ethyl silicate polymer | 1.00 |
| CAPA 2043 | Linear Polycaprolactone diol | 0.38 |
| | (MW = 400) | |
| Water | | 52.74 |
| Poval 18-88 | Polyvinyl alcohol | 0.40 |
| Finnfix 5 | Sodium Carboxymethylcellulose | 0.15 |
| Lupasol PR8515 | Polyethyleneimine | 0.20 |
| NaHCO₃ | Sodium bicarbonate | 0.33 |
| Luviquat PQ11 | Polyquaternium D11 | 10.00 |
| | (Deposition additive) | |
| Urea | | 2.75 |
| Keltrol RD | Xanthan gum | 0.20 |
| | TOTAL | 100.00 |

Microcapsules 1 according to the invention were formed using the materials of Table 2 as follows. Pre-mix I was prepared from 5.20 g of Poval 18-88, 567.58 g of water and 1.95 g of Finnfix 5 with heating, if necessary, until completely dissolved. Pre-mix II was prepared from 386.10 g of Fragrance 1 (containing standard fragrance compounds and solvents available from Iberchem), 27.95 g of Takenate D131N (ex Mitsui Chemicals), 13.00 g of Wacker TES40 (ex Wacker), added dropwise and 4.94 g of CAPA 2043 (ex Perstorp). The two pre-mixes I & II were combined and emulsified with the help of an Ultraturrax T25 at room temperature at a speed of 9000 rpm. The pH of the emulsion was then adjusted to 2.5 using aqueous hydrochloric acid solution (10% strength by weight). Then, at 35° C. and with stirring at 150 l/min, a solution of 2.60 g of Lupasol PR8515 (polyethyleneimine, ex BASF) in a sodium bicarbonate solution at 7% in water was added to the emulsion over the course of 2 hours. The reaction mixture was then subjected to the following temperature program: heating to 55° C. after those 2 hours, maintaining this temperature for 2 hours, then 3 hours at 80° C. After this time, add the 130.0 g of Luviquat PQ11 (ex BASF) and allow to mix for 15 minutes with Ultraturrax T25 at 8000 rpm. Then prepare a premix of urea and water (35.75 g of each) with heat at 60° C., add it to the mixture and when it dissolves add 2.6 g of Keltrol RD to the mixture with stirring and turn off the heating plate. The mixture was then cooled to room temperature. Finally, the pH of the produced microcapsule slurry was adjusted to 7.5 using aqueous sodium hydroxide solution or hydrochloric acid solution.

The produced microcapsule slurry according to the invention will be referred to as Microcapsules 1.

Microcapsules 2 & 3 according to the invention were synthesised using the materials listed in Table 2 using the process described in this Example with the following material changes:
a) Microcapsules 2 were made by replacing CAPA 2043 with the same weight of CAPA 2101 (1000 Mw linear polycaprolactone diol)
b) Microcapsules 3 were made by replacing CAPA 2043 with the same weight of CAPA 3050 (500 Mw polycaprolactone triol)

Microcapsules 4 according to the invention were synthesised using the materials listed in Table 3 using the process described in this Example with the poylvinyl alcohol (Poval 18-88) of Microcapsules 1 being replaced with glucose monohydrate.

TABLE 3
Polymerization system for Microcapsules 4

| | Amount |
|---|---|
| | (wt % of total |
| | polymerization |
| Ingredient name | system) |
| FRAGRANCE 1 | 29.70 |
| Takenate D131N | 1.79 |
| Wacker TES40 | 1.00 |
| Polycaprolactone diol (PCLD) CAPA 2043 | 0.38 |
| Glucose monohydrate CAS 5996-10-1 | 1.92 |
| Finnfix 5 (CMC) | 0.15 |
| Lupasol PR 8515 | 0.20 |
| NaHCO3 solution 7% | 4.66 |
| Luviquat PQ 11 | 10.00 |
| Water | To 100.00 |
| Keltrol RD | 0.30 |
| TOTAL | 100.00 |

Microcapsules 5 according to the invention were synthesised using the materials listed in Table 4 using the process described in this Example with the CAPA 2043 of Microcapsules 1 being replaced with CAPA 3050 and the Takenate D131N polyisocyanate being replaced with Takenate D110N.

TABLE 4
Polymerization system for Microcapsules 5

| | Amount |
|---|---|
| | (wt % of total |
| | polymerization |
| Ingredient name | system) |
| FRAGRANCE 1 | 29.70 |
| Polycaprolactone triol CAPA 3050 | 0.38 |
| Takenate D110N | 2.62 |
| Wacker TES40 | 1.00 |
| Finnfix 5 (CMC) | 0.15 |
| Lupasol PR 8515 | 0.20 |
| NaHCO3 solution 7% | 4.66 |
| Luviquat PQ 11 | 10.00 |
| Urea | 2.75 |
| Water | To 100.00 |
| Keltrol RD | 0.30 |
| TOTAL | 100.00 |

Microcapsules 6 according to the invention were synthesised using the materials listed in Table 5 using the process described in this Example, with the Keltrol RD being replaced with polyquaternium 10 and with the poylvinyl alcohol (Poval 18-88) of Microcapsules 1 being replaced with glucose monohydrate.

TABLE 5
Polymerization System for Microcapsules 6

| | Amount |
|---|---|
| | (wt % of total |
| | polymerization |
| Ingredient name | system) |
| FRAGRANCE 1 | 29.70 |
| Polycaprolactone diol (PCLD) CAPA 2043 | 0.38 |
| Takenate D110N | 2.62 |
| Wacker TES40 | 1.00 |
| Glucose monohydrate CAS 5996-10-1 | 1.92 |
| Finnfix 5 (CMC) | 0.15 |
| Lupasol PR 8515 | 0.20 |
| NaHCO3 solution 7% | 4.66 |
| Luviquat PQ 11 | 5.00 |
| Polyquaternium 10 | 0.77 |
| Water | 10.37 |
| TOTAL | 100.00 |

### Example 2

Comparative Microcapsules A from Example A and Microcapsules 1 from Example 1 were compared as follows. Particle size analysis was performed as described in the Test Methods herein to obtain D10, D50 and D90 volume mean diameters of the microcapsules. The results are given in Table 5.

TABLE 5
Particle Size analysis

| Sample | D10 (μm) | D50 (μm) | D90 (μm) |
|---|---|---|---|
| Comparative Microcapsules A | 3.20 | 9.77 | 18.4 |
| Microcapsules 1 | 2.16 | 6.44 | 12.7 |

It can be seen from Table 5 that Microcapsules 1 of the invention have smaller D10, D50 and D90 volume mean diameters than Comparative Microcapsules A.

### Example 3

Microcapsules 1 from Example 1 and Comparative Microcapsules A from Example A were compared for fragrance release performance in liquid laundry detergent as described in the Test Methods herein. The results are given in Table 6.

TABLE 6
Fragrance release performance in Liquid Laundry Detergent

| Laundry Detergent | Before rubbing | After rubbing |
|---|---|---|
| Microcapsules 1 | 2 | 8 |
| Comparative Microcapsules A | 2 | 6 |

Surprisingly, it can be seen from Table 6 that Microcapsules 1 according to the invention have a better fragrance release performance after rubbing than Comparative Microcapsules A in liquid laundry detergent. Without being bound by theory, this advantage may be attributed to the presence of polycaprolactone polyol in the shell components of Microcapsules 1.

### Example 4

Microcapsules 1 & 3 from Example 1 were compared for fragrance release performance in liquid laundry detergent & fabric softener as described in the Test Methods herein. The results are given in Tables 7 & 8.

TABLE 7
Fragrance release performance in Liquid Laundry Detergent

| Laundry Detergent | Before rubbing | After rubbing |
|---|---|---|
| Microcapsules 1 | 1 | 8 |
| Microcapsules 3 | 1 | 9 |

TABLE 8
Fragrance release performance in Fabric Softener

| Laundry Detergent | Before rubbing | After rubbing |
|---|---|---|
| Microcapsules 1 | 3 | 9 |
| Microcapsules 3 | 3 | 10 |

Surprisingly, it can be seen from Tables 7 & 8 that Microcapsules 3 performed better that Microcapsules 1. Without being bound by theory, this advantage may be attributed to the presence of polycaprolactone triol in the shell components of Microcapsules 3.

### Example 5

Comparative Microcapsules A from Example A and Microcapsules 1 & 4 from Example 1 were compared for fragrance release performance in liquid laundry detergent & fabric softener as described in the Test Methods herein. The results are given in Tables 9 & 10.

TABLE 9
Fragrance release performance in Liquid Laundry Detergent

| Laundry Detergent | Before rubbing | After rubbing |
|---|---|---|
| Microcapsules 1 | 2 | 7 |
| Microcapsules 4 | 2 | 9 |
| Comparative Microcapsules A | 2 | 5 |

TABLE 10
Fragrance release performance in Fabric Softener

| Laundry Detergent | Before rubbing | After rubbing |
|---|---|---|
| Microcapsules 1 | 2 | 8 |
| Microcapsules 4 | 2 | 10 |
| Comparative Microcapsules A | 2 | 7 |

Surprisingly, it can be seen from Tables 9 & 10 that Microcapsules 1 & 4 performed better that Comparative Microcapsules A. Without being bound by theory, this advantage may be attributed to the presence of polycaprolactone polyol in the shell components of Microcapsules 1 & 4 and the presence of glucose monohydrate in the shell components of Microcapsules 4.

### Example 6

Microcapsules 1 & 5 from Example 1 were compared for fragrance release performance in liquid laundry detergent & fabric softener as described in the Test Methods herein. The results are given in Tables 11 & 12.

TABLE 11
Fragrance release performance in Liquid Laundry Detergent

| Laundry Detergent | Before rubbing | After rubbing |
|---|---|---|
| Microcapsules 1 | 2 | 7 |
| Microcapsules 5 | 3 | 9 |

TABLE 12
Fragrance release performance in Fabric Softener

| Laundry Detergent | Before rubbing | After rubbing |
|---|---|---|
| Microcapsules 1 | 2 | 8 |
| Microcapsules 5 | 2 | 9 |

Surprisingly, it can be seen from Tables 11 & 12 that Microcapsules 5 performed better that Microcapsules 1. Without being bound by theory, this advantage may be attributed to the presence of polycaprolactone triol in the shell components of Microcapsules 5.

### Example 7

Comparative Microcapsules A from Example A and Microcapsules 1 from Example 1 were compared for biodegradability of the capsule shells under OECD 301F as described in the Test Methods herein. The shells of the capsules were isolated and prepared for the OECD 301F test using the following procedure:
1) Filtration to remove the water- and water-soluble components
2) Washing the filtrate several times
3) Drying the filtrate using infrared dryer
4) Grinding the solid material obtained, to break the capsules
5) Washing with solvent to remove the fragrance.
6) Repeating several times steps 3 to 5 until all the fragrance is removed
7) Finally the solid wall material is reconstituted at 5% in sterile water for biodegradability testing.

Under the OECD 301F test at 28 days, Comparative Microcapsules A had a mean degradation of 89% and Microcapsules 1 had a mean degradation of 95%. This shows that Microcapsules 1 are more biodegradable that Comparative Microcapsules A.

It is to be understood that the invention is not to be limited to the details of the above embodiments, which are described by way of example only. Many variations are possible.

1. Microcapsules comprising a hydrophobic core within a polymeric shell, wherein:
a) the polymeric shell is formed from shell components comprising:
i) a polyisocyanate, wherein the polyisocyanate is selected from xylylene diisocyanate (XDI) and oligomers, adducts and derivatives thereof;
ii) a polycaprolactone polyol comprising from 2 to 4 free hydroxyl groups;
iii) a polyethyleneimine;
iv) optionally, other shell components; and
b) the hydrophobic core comprises an active material.

2. The microcapsules according to claim 1 wherein the polymeric shell further comprises an alkyl silicate.

3. The microcapsules according to claim 2 wherein the polymeric shell comprises 7.5% to 25% of the alkyl silicate and 20% to 75% of the polyisocyanate, by weight, on the basis of the total weight of shell components in the microcapsules.

4. (canceled)

5. The microcapsules according to claim 1 wherein the polymeric shell comprises only one polyisocyanate.

6. The microcapsules according to claim 1 wherein the polymeric shell comprises 2.5% to 30% of the polycaprolactone polyol, by weight, on the basis of the total weight of shell components in the microcapsules and the polycaprolactone polyol has a molecular weight in the range from 200 to 10,000 g/mol.

7. The microcapsules according to claim 1 wherein the polycaprolactone polyol is formed from caprolactone monomer and a diol or triol initiator.

8. (canceled)

9. The microcapsules according to claim 1 wherein the polymeric shell comprises 1% to 10% of the polyethyleneimine, by weight, on the basis of the total weight of shell components in the microcapsules.

10. The microcapsules according to claim 1 wherein the polyethyleneimine has a branched structure, a molecular weight in the range from 500 to 5,000 g/mol, and is cationic.

11. (canceled)

12. (canceled)

13. The microcapsules according to claim 1 wherein the microcapsules do not comprise a wax with a melting point above 40° C.

14. (canceled)

15. The microcapsules according to claim 1 wherein the microcapsules do not comprise an aminoplast resin.

16. The microcapsules according to claim 1 further comprising a deposition additive on the surface of the microcapsules, preferably comprising a hydrolysed protein.

17. A slurry comprising the microcapsules according to claim 1, water and at least one surfactant.

18. A home care formulation comprising the microcapsules according to claim 1 and at least one additional home care ingredient.

19. (canceled)

20. A personal care formulation comprising the microcapsules according to claim 1 and at least one additional personal care ingredient, wherein the personal care formulation is selected from hair care products, skin care products, cosmetics, personal care cleansers, deodorants and anti-perspirants.

21. (canceled)

22. A process of producing the microcapsules according to claim 1, wherein the process comprises the steps of:
a) forming a polymerization system comprising an aqueous phase and a dispersed oil phase, wherein the oil phase comprises the active material, the polyisocyanate shell component and the polycaprolactone polyol shell component;
b) reacting the shell components by adding the polyethyleneimine shell component to the aqueous phase to form microcapsules comprising a core of the oil phase within the polymeric shell;
c) optionally, adding a deposition additive to the surface of the microcapsules; and
d) optionally, neutralizing the microcapsules using a metal hydroxide or inorganic acid.

23. The process according to claim 22 wherein the polymerization system further comprises a sugar or a sugar alcohol and carboxymethylcellulose.

24. The process according to claim 22 wherein the polymerization system does not comprise polyvinyl alcohol.

25. (canceled)

26. The process according to claim 22, comprising step c) adding a deposition additive to the surface of the microcapsules, wherein the deposition additive is a cationic polymer.

27. (canceled)

28. The process according to claim 22 which comprises the step of adding urea to the polymerization system after the microcapsules have been formed.

29. (canceled)

30. Microcapsules obtained by a process according to claim 22.
